(19) United States
(12) Plant Patent Application Publication
(10) Pub. No.: US 2008/0141404 P1
(43) Pub. Date: Jun. 12, 2008

(54) Strawberry plant named 'PS-5298'

(75) Inventors: Stephen M. Ackerman, Salinas, CA (US); Steven D. Nelson, Watsonville, CA (US); Michael D. Nelson, Watsonville, CA (US)

Correspondence Address: FOLEY AND LARDNER LLP;SUITE 500, 3000 K STREET NW, WASHINGTON, DC, 20007, US

(73) Assignees: PLANT SCIENCES, INC.; BERRY R&D, INC

(21) Appl. No.: 11/710,528

(22) Filed: Feb. 26, 2007

Related U.S. Application Data
(60) Provisional application No. 60/853,002, filed on Oct. 20, 2006.

(51) Int. Cl. A01H 5/00

(52) U.S. Cl. PLT/208

(57) ABSTRACT

This invention relates to a new and distinct variety of strawberry plant named ‘PS-5298’. This new strawberry plant named ‘PS-5298’ is primarily adapted to the growing conditions of the central coast of California, and is primarily characterized by its large plant size; medium to large fruit size; medium to red fruit color; spreading to reflexed calyx segments; medium to strong skin firmness; good fruit production with good flavor; light to medium green foliage with glossy texture and medium to strong interveinal blistering; and medium to large foliage, concave to flat in shape.

### CROSS REFERENCE TO RELATED APPLICATION

This application claims priority under 35 U.S.C. § 119(e) of the U.S. Provisional Patent Application No. 60/853,002 filed Oct. 20, 2006.

### LATIN NAME OF THE GENUS AND SPECIES OF THE PLANT CLAIMED

Fragaria ananassa

### VARIETY DENOMINATION

‘PS-5298’

### BACKGROUND OF THE INVENTION

The present invention relates to a new and distinct strawberry variety designated as ‘PS-5298’. This new variety is a result of a controlled cross made by the inventors, Stephen M. Ackerman, Steven D. Nelson, and Michael D. Nelson, in 1997 between strawberry variety ‘PS-592’ (patented, U.S. Plant Pat. No. 9,903) and strawberry variety designated ‘Aromas’ (patented, U.S. Plant Pat. No. 10,451). The variety is botanically known as Fragaria ananassa.

The seedling resulting from the aforementioned cross was asexually propagated by stolons in a nursery located in Siskiyou County, California, and was subsequently selected by the inventors from a controlled breeding plot in Salinas, Calif., in 1999. After its selection, the new variety was further asexually propagated by stolons in both Siskiyou County, California and San Joaquin County, California. The new variety was extensively tested over the next several years in fruiting fields of Salinas, Calif. This propagation has demonstrated that the combination of traits disclosed herein as characterizing the new variety are fixed and remain true to type through successive generations of asexual reproduction.

### BRIEF SUMMARY OF THE INVENTION

‘PS-5298’ is primarily adapted to the climate and growing conditions of the central coast of California. This region provides the necessary winter temperatures required for it to produce a strong vigorous plant and to remain in fruit production from April through November. The nearby Pacific Ocean provides the needed humidity and moderate temperatures to maintain fruit quality during the spring and summer production months.

The following traits have been repeatedly observed and are determined to be unique characteristics of ‘PS-5298’, which in combination distinguish this strawberry plant as a new and distinct variety:
1. Large plant size;
2. Medium to large fruit size;
3. Medium to red fruit color;
4. Spreading to reflexed calyx segments;
5. Medium to strong skin firmness;
6. Good fruit production with good flavor;
7. Light to medium green foliage with glossy texture and medium to strong interveinal blistering; and
8. Medium to large foliage, concave to flat in shape.

The strawberry varieties that are believed to be most closely related to the new strawberry variety ‘PS-5298’ are the parental strawberry variety ‘PS-592’ (patented, U.S. Plant Pat. No. 9,903) and the strawberry variety ‘PS-1150’ (patented, U.S. Plant Pat. No 10,780).

In comparison to the similar strawberry varieties ‘PS-592’ and ‘PS-1150’, the new strawberry variety ‘PS-5298’ differs by the following combination of characteristics as described in Table 1:

TABLE 1

| | | | ‘PS-592’ | ‘PS-1150’ |
|---|---|---|---|---|
| | | | (US | (US |
| Characteristic | | ‘PS-5298’ | PP9,903) | PP10,780) |
| 1. | Color of mature fruit | Red | Red to red | Red |
| | | | orange | |
| 2. | Average fruit weight | 23.5 | 23.1 | 17.6 |
| | (gm) | | | |
| 3. | Marketable yield (gm/plt) | 1,362 | 1,392 | 1,010 |
| 4. | Attitude of calyx | Spreading to | Spreading to | Spreading to |
| | | reflexed | reflexed | collapsing |
| 5. | Firmness of skin | Medium to | Medium | Strong |
| | | strong | | |
| 6. | Time of first harvest | Early | Early | Late |
| 7. | Plant size | Large | Large | Medium |
| 8. | Foliage color (upper | Light to | Medium | Medium |
| | side) | medium | green | green |
| | | green | | |
| 9. | Interveinal leaf blistering | Medium to | Medium to | Weak to |
| | | strong | strong | medium |
| 10. | Foliage glossiness | Medium to | Strong | Weak |
| | | strong | | |
| 11. | Foliage size | Medium to | Large | Small |
| | | large | | |
| 12. | Fruiting truss position | Level with to | Level with to | Above |
| | relative to foliage | beneath | beneath | |

For identification a series of molecular markers have been determined for this new variety.

### BRIEF DESCRIPTIONS OF THE DRAWINGS

The accompanying color photographs illustrate the overall appearance of typical specimens of the new strawberry variety ‘PS-5298’, at various stages of development as true as it is reasonably possible with color reproductions of this type. Color in the photographs may differ slightly from the color value cited in the botanical description which accurately describe the color of ‘PS-5298’. The depicted plant and plant parts of the new strawberry variety ‘PS-5298’ were taken in Salinas, Calif., and are approximately 8 to 9 months old.

FIG. 1 shows typical fruiting field characteristics taken in the month of June, 2006;

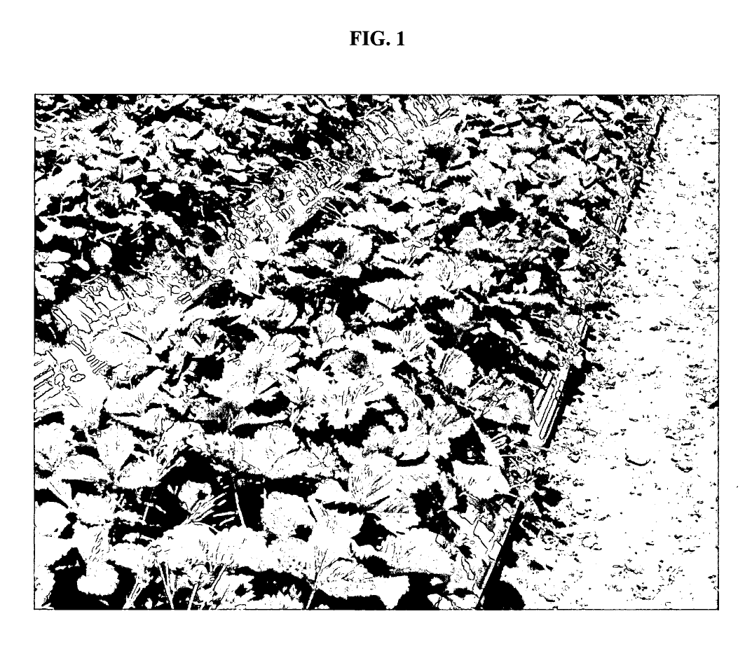

FIG. 2. shows a close-up view of typical leaf structure taken in the month of June, 2006;

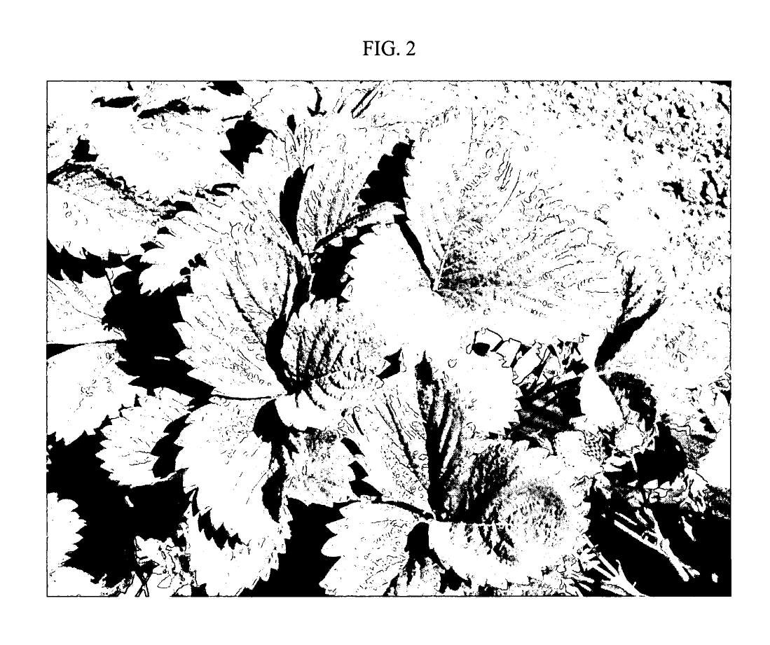

FIG. 3 shows typical mature and immature field fruit taken in the month of June, 2006; and

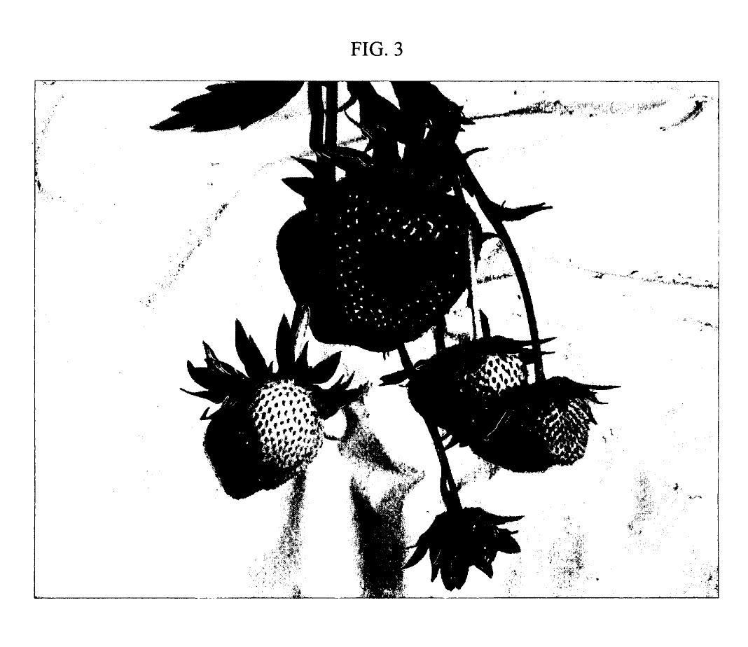

FIG. 4 shows a close-up view of fruit taken in the month of July, 2006.

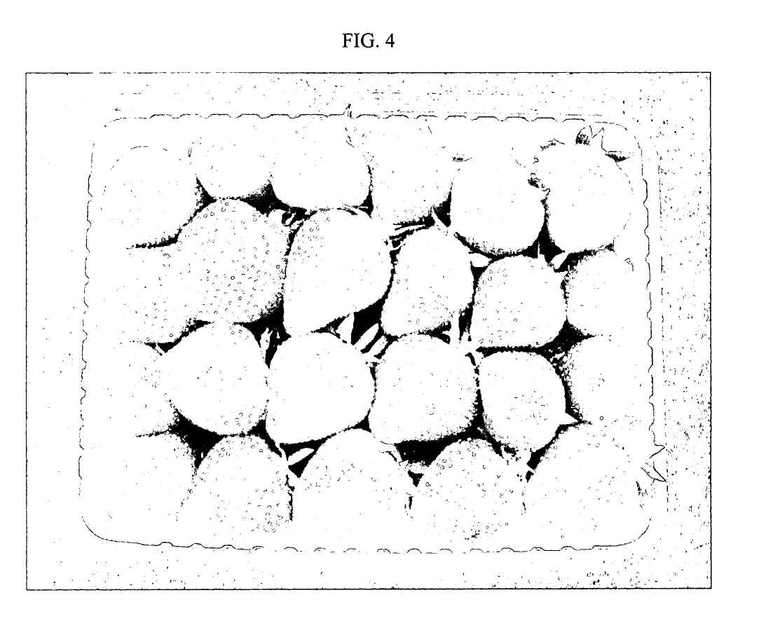

### DETAILED BOTANICAL DESCRIPTION

‘PS-5298’ has not been observed under all possible environmental conditions. The characteristics of the new variety may vary in detail, depending upon variations in environmental factors, including weather (temperature, humidity and light intensity), day length, soil type and location.

The aforementioned photographs, together with the following description of the new variety ‘PS-5298’, unless otherwise noted, is based on observations taken during the 2006 growing season in Salinas, Calif. These measurements and ratings were taken from plants of ‘PS-5298’ dug from a high-elevation nursery located in Siskiyou County, California, during the middle of October 2005 and planted approximately 3 weeks later in Salinas, Calif. The approximate age of the observed plants is 7 to 10 months. Yield observations and fruit quality characteristics are averaged from four years of data collected from the 2003 through 2006 growing seasons. Flower measurements and characteristics are from secondary flowers unless otherwise noted. Fruit characteristics and measurements are from secondary fruit unless otherwise noted.

Color termninology where noted follows the Munsell Book of Colors, Munsell Color, Baltimore, Md. (1976).

The following Tables 2-8 describe fruit, plant, stolon, foliage, fruiting truss, flower and pest/disease characteristics of the new strawberry ‘PS-5298’ in comparison to the similar strawberry varieties ‘PS-592’ and ‘PS-1150’.

TABLE 2
FRUIT CHARACTERISTICS

| | | ‘PS-592’ | ‘PS-1150’ |
|---|---|---|---|
| Characteristic | ‘PS-5298’ | (US PP9,903) | (US PP10,780) |
| Color of mature fruit | 7.5R 3/10 to 4/10 | 7.5R 3/12 to 4/12 | 7.5R 4/10 to 3/8 |
| | Red | Red to orange red | Red |
| Color of internal flesh | 7.5R 4/10 to 4/12 | 7.5R 5/10 to 4/10 | 7.5R 4/10 to 4/12 |
| | Medium red | Light red | Medium red |
| Length (cm) | 4.9 | 4.8 | 4.2 |
| Width (cm) | 4.3 | 4.3 | 3.7 |
| Ratio length/width | 1.15 | 1.13 | 1.14 |
| | Slightly longer | Slightly longer | Slightly longer |
| | than broad | than broad | than broad |
| Calyx diameter (cm) | 5.0 | 5.0 | 4.2 |
| Average weight (gm) | 23.5 | 23.1 | 17.6 |
| Size | Medium to large | Medium to large | Small |
| Achene color | 5Y 6/8 to 7.5R 3/8 | 5Y 6/8 to 7.5R 3/8 | 5Y 6/8 to 7.5R 3/8 |
| Achenes per berry | 285 | 304 | 293 |
| Achene weight (mg) | 0.64 | 0.61 | 0.56 |
| Marketable yield (gm/plt) | 1,362 | 1,392 | 1,010 |
| Predominant shape | Conical | Conical | Conical |
| Difference in shapes | Moderate | Moderate | Slight |
| between primary and | | | |
| secondary fruit | | | |
| Band without achenes | Absent or | Absent or | Absent or |
| | very narrow | very narrow | very narrow |
| Unevenness of surface | Weak | Weak | Absent or |
| | | | very weak |
| Evenness of color | Slightly uneven | Slightly uneven | Even |
| | to even | to even | |
| Glossiness | Medium to strong | Medium to strong | Strong |
| Insertion of achenes | Level with surface | Level with surface | Level with |
| | | | surface |
| Insertion of calyx | Level with to | Level with to | Level with to |
| | above fruit | above fruit | in the basin |
| Attitude of the calyx | Spreading to | Spreading to | Spreading to |
| | reflexed | reflexed | collapsing |
| Size of calyx in relation to | Slightly larger | Slightly larger | Slightly larger |
| fruit diameter | | | |
| Adherence of calyx | Strong | Strong | Strong |
| Firmness of skin | Medium to strong | Medium | Strong |
| Firmness of flesh | Medium to firm | Medium | Medium to firm |
| Distribution of red color | Marginal and central | Marginal and central | Marginal and |
| of the flesh | | | central |
| Hollow center expression | Moderate | Moderate to strong | Moderate to strong |
| Flavor | Good | Very good | Fair |
| Soluble solids (% brix) | 8.0 | 8.0 | 7.4 |
| Time of first flowering | Early | Early | Late |
| Time of first harvesting | Early | Early | Late |
| Harvest period | Late March to | Late March to | Mid April to |
| | early December | early December | early December |
| Type of bearing | Partially remontant | Partially remontant | Partially remontant |

TABLE 3
PLANT CHARACTERISTICS

| | | ‘PS-592’ | ‘PS-1150’ |
|---|---|---|---|
| Characteristic | ‘PS-5298’ | (US PP9,903) | (US PP10,780) |
| Height (cm) | 30.8 | 31.0 | 30.0 |
| Spread (cm) | 36.8 | 35.1 | 28.8 |
| Crowns/plant | 5.3 | 5.0 | 5.0 |
| Size | Large | Large | Medium |
| Habit | Globose | Globose | Globose |
| Density | Medium | Medium to open | Medium |
| Vigor | Strong | Strong | Medium |

TABLE 4
STOLON CHARACTERISTICS

| | | ‘PS-592’ | ‘PS-1150’ |
|---|---|---|---|
| Characteristic | ‘PS-5298’ | (US PP9,903) | (US PP10,780) |
| Average number | 1.1 | 1.2 | 0.8 |
| per plant | | | |
| fruiting field | Few to medium | Few to medium | Few |
| Anthocyanin | 7.5R 5/6 to 6/6 | 7.5R 5/6 to 5/4 | 7.5R 4/6 to 5/6 |
| coloration | | | |
| Anthocyanin intensity | Weak | Medium | Medium to |
| | | | strong |
| Diameter at bract | 4.7 | 4.4 | 4.2 |
| (mm) | Thick | Thick | Medium |
| Pubescence | Medium to | Medium | Medium to |
| | strong | | strong |

TABLE 5
FOLIAGE CHARACTERISTICS

| | | ‘PS-592’ | ‘PS-1150’ |
|---|---|---|---|
| Characteristic | ‘PS-5298’ | (US PP9,903) | (US PP10,780) |
| Foliage: | | | |
| Color of upper surface | 7.5GY 4/4 to 3/4 | 7.5GY 4/4 to 3/4 | 7.5GY 4/4 to 3/4 |
| | Light to medium | Medium green | Medium green |
| | green | | |
| Color of under side | 5GY 5/4 to 6/4 | 5GY 5/4 to 6/4 | 5GY 5/4 to 6/4 |
| | Light to medium | Light grey green | Medium grey green |
| | grey green | | |
| Petiole length (cm) | 23.1 | 21.1 | 19.0 |
| Petiole diameter (mm) | 3.8 | 4.7 | 3.1 |
| Petiolule length (mm) | 14.0 | 15.7 | 9.3 |
| Stipule length (mm) | 20.7 | 20.8 | 14.5 |
| Stipule width (mm) | 8.3 | 8.5 | 8.4 |
| Size bract leaflets | Small | Small to medium | Small |
| Shape in cross section | Slightly concave to | Slightly concave | Slightly concave |
| | flat | | to slightly convex |
| Interveinal blistering | Medium to strong | Medium to strong | Weak to medium |
| Glossiness | Medium to strong | Strong | Weak |
| Number of leaflets | 3 | 3 | 3 to 4 |
| Petiole pubescence | Moderate to sparse | Moderate to sparse | Moderate to |
| | | | sparse |
| Petiole attitude of hairs | Strongly outward | Strongly outward | Strongly outward |
| Stipule anthocyanin | Weak | Medium | Absent or very |
| coloration | | | weak |
| Stipule color | Light to medium | Light to medium | Light to medium |
| | green | green | green |
| Terminal Leaflet: | | | |
| Length (cm) | 9.3 | 9.5 | 7.2 |
| Width (cm) | 7.6 | 8.0 | 6.4 |
| Length/width ratio | 1.22 | 1.19 | 1.13 |
| | Much longer than | Much longer than | Longer than |
| | broad | broad | broad |
| Serrations/leaf | 20.4 | 20.7 | 19.3 |
| Size | Medium to large | Large | Small |
| Shape of base | Acute | Acute | Obtuse |
| Shape of teeth | Obtuse | Obtuse | Obtuse |

TABLE 6
FRUITING TRUSS CHARACTERISTICS

| | | ‘PS-592’ | ‘PS-1150’ |
|---|---|---|---|
| Characteristic | ‘PS-5298’ | (US PP9,903) | (US PP10,780) |
| Length (cm) | 37.4 | 37.3 | 38.1 |
| Position relative to | Level with to | Level with to | Above |
| foliage | beneath | beneath | |
| Pubescence | Medium | Medium | Medium |
| Anthocyanin intensity | Light | Light | Moderate |
| Attitude at first pick | Prostrate | Prostrate | Prostrate |

TABLE 7
FLOWER CHARACTERISTICS

| | | ‘PS-592‘ | ‘PS-1150’ |
|---|---|---|---|
| Characteristic | ‘PS-5298’ | (US PP9,903) | (US PP10,780) |
| Petal color | N9.5/90.0% R | N9.5/90.0% R | N9.5/90.0% R |
| | to | to | to |
| | N9.25/84.2% R | N9.25/84.2% R | N9.25/84.2% R |
| | White | White | White |
| Corolla diameter (mm) | 32.3 | 30.6 | 30.5 |
| | Large | Large | Medium |
| Calyx diameter (mm) | 39.9 | 38.8 | 34.0 |
| Petal length (mm) | 12.4 | 12.1 | 12.5 |
| Petal width (mm) | 12.6 | 11.5 | 10.8 |
| Petal length/width | 0.98 | 1.05 | 1.15 |
| ratio | | | |
| Petals/flower | 5.9 | 6.0 | 6.2 |
| Sepal length (mm) | 16.0 | 15.7 | 13.1 |
| Sepal width (mm) | 6.2 | 6.2 | 5.4 |
| Sepal length/width | 2.57 | 2.54 | 2.42 |
| ratio | | | |
| Sepals/flower | 11.8 | 11.9 | 12.4 |
| Size of calyx relative | Larger | Larger | Larger corolla |
| to | | | |
| Size of inner calyx | Smaller to | Smaller to | Smaller to |
| relative to outer calyx | same size | same size | same size |
| Relative position of | Overlapping | Overlapping | Overlapping |
| petals | | | |

TABLE 8
PEST AND DISEASE REACTIONS

| | | ‘PS-592’ | ‘PS-1150’ |
|---|---|---|---|
| Characteristic | ‘PS-5298’ | (US PP9,903) | (US PP10,780) |
| Two spotted spider mite | Moderately | Moderately | Moderately |
| | susceptible | susceptible | susceptible |
| Lygus bug | Susceptible | Susceptible | Susceptible |
| Flower thrips | Moderately | Moderately | Moderately |
| | susceptible | susceptible | susceptible |
| Powdery mildew | Moderately | Moderately | Moderately |
| | susceptible | susceptible | susceptible |
| Botrytis fruit rot | Moderately | Moderately | Moderately |
| | Susceptible | susceptible | susceptible |
| Angular leaf spot | Moderately | Moderately | Moderately |
| | susceptible | susceptible | susceptible |
| Virus complex | Unknown | Unknown | Unknown |

We claim:

1. A new and distinct strawberry plant named ‘PS-5298’, as herein described and illustrated by the characteristics set forth above.
